(19) United States
(12) Patent Application Publication
(10) Pub. No.: US 2012/0129946 A1
(43) Pub. Date: May 24, 2012

(54) MATERIALS AND METHODS FOR TREATING VIRAL INFECTIONS

(76) Inventor: Jess Gilbert Thoene, Ann Arbor, MI (US)

(21) Appl. No.: 13/302,620

(22) Filed: Nov. 22, 2011

Related U.S. Application Data
(63) Continuation of application No. PCT/US2010/035785, filed on May 21, 2010.
(60) Provisional application No. 61/180,840, filed on May 23, 2009.

(51) Int. Cl. A61K 31/131; A61P 31/20; A61P 31/22; A61P 31/14; A61P 31/12; A61P 31/16

(52) U.S. Cl. 514/665

(57) ABSTRACT

The subject invention provides materials and methods for the prevention and/or treatment of viral infections. In a preferred embodiment, a cysteamine compound is administered via pulmonary administration to a subject to treat an influenza virus infection.

### CROSS-REFERENCE TO A RELATED APPLICATION

This application is a continuation of International Application No. PCT/US2010/035785, filed May 21, 2010; which claims the benefit of U.S. provisional application Ser. No. 61/180,840, filed May 23, 2009, all of which are hereby incorporated herein by reference in their entirety.

### BACKGROUND OF THE INVENTION

A virus is a small infectious agent consisting of nucleic acid (RNA or DNA) enclosed in a protein coat. Viruses can only replicate by infecting a susceptible host cell and directing the host cell machinery to produce more viruses. Glycoproteins (located in the protein coat) mediate the adsorption to, and the penetration of, the virus into susceptible host cells.

Important virus families of the DNA type (also classified as Classes I and II viruses—See Harvey, L. et al., Molecular Cell Biology, Fourth Edition, W.H. Freeman and Company (2000)) include adenoviridae, herpesviridae, poxviridae, papovaviridae, densovirinae, and parvovirinae. Virus families typically classified of the RNA type (also classified as Classes III-VI, See Molecular Cell Biology) include birnaviridae, reoviridae, astoviridae, arterivirus, caliciviridae, coronaviridae, flaviviridae, picornaviridae, togaviridae, polioviruses, bornaviridae, filoviridae, paramyxovirinae, pneumovirinae, rhabdoviridae, bunyaviridae, and orthomyxoviridae.

Influenza, commonly known as the “flu,” is a contagious disease that is caused by the influenza virus, classified in the orthomyxoviridae family. There are three known influenza-type viruses which affect human beings: Influenza A, B and C. Influenza A viruses have been isolated from many animal species in addition to humans, while the influenza B and C viruses have been found to infect mainly humans.

Influenza viruses are enveloped viruses containing negative single-stranded RNA's which are segmented and encapsidated. The influenza virus envelope is characterized by the presence of two surface glycoproteins: hemagglutinin and neuraminidase. The influenza A and B virions are pleomorphic and are usually 80-120 nm in diameter. The influenza C virion has many distinctive properties and is thus distinguished from the closely related A and B virions.

Influenza viruses attack the respiratory tract in humans (i.e., nose, throat, and lungs). For example, infection with influenza A or B often can cause a highly contagious, acute respiratory illness. Influenza infection usually includes the following symptoms: fever, headache, tiredness (can be extreme), dry cough, sore throat, nasal congestion, and body aches.

It is estimated that millions of people in the United States13 about 10% to 20% of U.S. residents—get influenza each year. The majority of this population generally recovers in one to two weeks. In some cases, however, complications can arise from an influenza infection. An average of about 36,000 people per year in the United States die from influenza, and 114,000 per year have to be admitted to the hospital as a result of the infection.

Furthermore, the spread of influenza virus through a population can result in epidemics. High rates of mortality were observed due to influenza infection during the influenza epidemics of 1957, 1968 and 1977 (Fields Virology, Second Edition, Volume 1, pp. 1075-1152 (1990)). Periodically, the influenza virus causes a worldwide epidemic. For example, the influenza pandemic of 1918 reportedly caused about 20 million deaths worldwide and about 500,000 deaths in the United States (Medical Microbiology, Fourth Edition, University of Texas Medical Branch at Galveston (1996)).

There are many different subtypes of type A influenza viruses. These subtypes differ because of changes in certain proteins on the surface of the influenza A viruses (hemagglutinin [HA] and neuraminidase [NA] proteins). There are 16 known HA subtypes and 9 known NA subtypes of influenza A viruses. Many different combinations of HA and NA proteins are possible. Each combination represents a different subtype. “Human influenza virus” usually refers to those subtypes that spread widely among humans. There are only three known A subtypes of influenza viruses (H1N1, H1N2, and H3N2) currently circulating among humans. It is likely that some genetic parts of current human influenza A viruses came from birds or other animals originally. Influenza A viruses are constantly changing, and they might adapt over time to infect and spread among humans.

Avian influenza is an infection caused by avian (bird) influenza viruses. These influenza viruses occur naturally among birds. Wild birds carry the viruses in their intestines, but do not usually get sick from them. However, avian influenza is very contagious among birds and can make some domesticated birds, including chickens, ducks, and turkeys, very sick and kill them.

Symptoms of avian influenza in humans have ranged from typical human influenza-like symptoms (e.g., fever, cough, sore throat, and muscle aches) to eye infections, pneumonia, severe respiratory diseases (such as acute respiratory distress), and other severe and life-threatening complications.

Influenza A (H5N1) virus—also called “H5N1 virus”—is an influenza A virus subtype that occurs mainly in birds, is highly contagious among birds, and can be deadly to them. Infections with these viruses have also occurred in humans. Of the avian influenza viruses that have crossed the species barrier to infect humans, H5N1 has caused the largest number of detected cases of severe disease and death in humans.

Swine influenza (also called swine flu, hog flu, and pig flu) refers to influenza caused by those strains of influenza virus called swine influenza virus (SIV), that usually infects pigs. Rarely, strains of swine flu can pass from human to human. In humans, the symptoms of swine flu are similar to those of influenza and of influenza-like illness in general, namely chills, fever, sore throat, muscle pains, severe headache, coughing, weakness, and general discomfort.

The 2009 flu outbreak in humans, known as “swine flu”, is due to a new strain of influenza A virus subtype H1N1 that contains genes most closely related to swine influenza. The origin of this new strain is unknown. However, the World Organization for Animal Health (OIE) reports that this strain has not been isolated in pigs. This strain can be transmitted from human to human, and causes the normal symptoms of influenza.

Few methods are available for preventing an influenza infection and a cure has yet to be developed. Methods for preventing an influenza infection include vaccination and antiviral medications. Three antiviral drugs (arnantadine, rimantadine, and oseltamivir) have been approved in the United States and are commercially available for use in preventing or treating influenza virus disease. These compounds, however, are most effective when used prophylactically, which may allow influenza viruses to develop resistance to both compounds rapidly. See U.S. Pat. Nos. 3,352,912 and 3,152,180. Other compounds reported to have activity against influenza viruses have been disclosed in U.S. Pat. Nos. 6,271,373; 5,935,957; 5,821,243; 5,684,024; 3,592,934; 3,538,160; 3,534,084; 3,496,228; and 3,483,254, all of which are incorporated herein by reference.

There is a great need for new therapies for the treatment of viral diseases. Whereas there has been progress in developing a variety of therapies for the treatment of bacterial infections, there are few viable therapies for the treatment of viruses. As described above, antiviral drugs and vaccines are primary methods used in the prevention and/or treatment of influenza infections. However, these therapies can have substantial side effects based on their deleterious effects on host cell DNA replication or their effect on a limited number of viral infections. In addition, viruses are known to develop resistance to therapies, which causes a progressive decline in efficacy.

### BRIEF SUMMARY OF THE INVENTION

The subject invention provides materials and methods for treating subjects diagnosed with, or susceptible to, viral infections. In a preferred embodiment, viral infections are treated according to the subject invention by pulmonary administration of a cysteamine compound. The pulmonary administration may be accomplished via, for example, inhalation.

The present invention provides for the treatment and/or prevention of viral infections from Classes I through V viruses through the administration of a cysteamine compound to a subject. The subject invention is applicable to both human and animal health. In a specific embodiment, cysteamine, or a salt thereof; can be administered by inhalation to treat a subject infected with an influenza virus. The influenza virus may be, for example, avian flu or swine flu.

Specifically exemplified herein is the use of a cysteamine compound to treat and/or prevent an influenza virus infection. In accordance with the subject invention, administration of a cysteamine compound to a subject prior to acquiring the influenza virus can help protect the subject from influenza infection, or reduce symptoms related to the influenza virus infection. The cysteamine compound can be administered alone or concurrently with one or more other known agents that are used to treat/prevent a viral infection.

Preferably the compounds of the invention are administered to the respiratory tract by inhalation, insufflation or intranasal administration, or a combination thereof Administration to the respiratory tract may be achieved by, for example, an aerosol formulation in which the compound is provided in a pressurised pack with a suitable propellant. The dose of drug may be controlled by provision of a metered valve.

Alternatively the compounds may be provided in the form of a dry powder, for example a powder mix of the compound in a suitable powder base such as lactose, starch, starch derivatives such as hydroxypropylmethyl cellulose and polyvinylpyrrolidine (PVP).

In formulations intended for administration to the respiratory tract, including intranasal formulations, the compound will generally have a small particle size, for example of the order of five microns or less. Such a particle size may be obtained by means known in the art, for example by micronisation.

When desired, formulations adapted to give sustained release of the active ingredient may be employed.

In accordance with the subject invention, the daily dosage amount of a cysteamine compound administered to a subject prior to viral infection to protect the subject from viral infection can be about 10 mg to 3,000 mg. Preferably, a cysteamine compound is administered at about 50 mg to 1,500 mg per day. In a more preferred embodiment, about 200 mg to 900 mg of cysteamine hydrochloride is administered daily to a subject to prevent/treat the onset of an influenza (such as avian influenza virus, swine influenza virus, influenza A, influenza B, and influenza C or any mutants thereof) virus disease.

### BRIEF DESCRIPTION OF DRAWINGS

FIG. 1 shows cysteamine as a constituent of co-enzyme A.

FIG. 2 shows a metabolic pathway of cysteamine.

### DETAILED DISCLOSURE OF THE INVENTION

The subject invention provides materials and methods for treating subjects diagnosed with, or susceptible to, viral infections. In a preferred embodiment, viral infections are treated according to the subject invention by pulmonary administration of a cysteamine compound. The pulmonary administration may be accomplished via, for example, inhalation.

Specifically, the subject invention provides materials and methods for preventing a Class I-V viral infection; treating/ameliorating symptoms associated with Class I-V viral infections; and/or preventing/delaying the onset of complications associated with Class I-V viral infections.

### Definitions

The term “symptom(s)” as used herein, refers to common signs or indications that a subject is suffering from a specific condition or disease. For example, symptoms associated with a viral infection, as used herein, refer to common signs or indications that a subject is infected with a Class I-V virus. Influenza-related symptoms contemplated herein include, but are not limited to, fever, headache, exhaustion/fatigue, muscular aches, sore joints, irritated watering eyes, malaise, nausea and/or vomiting, shaking chills, chest pain, sneezing and respiratory symptoms (i.e., inflamed respiratory mucous membranes, substernal burning, nasal discharge, scratchy/sore throat, dry cough, loss of smell).

The terms “influenza,” “influenza virus,” or “flu,” as used herein, refer to an RNA virus of the Orthomyxoviridae family, including influenza A, influenza B, and influenza C, and mutants thereof. Influenza viruses contemplated herein include those viruses that have two antigenic glycosylated enzymes on their surface: neuraminidase and hemagglutinin. Various subtypes of influenza virus that can be treated using the materials and methods of the invention include, but are not limited to, the H1N1, H1N2, H2N2, H3N2, H3N8, H5N1, H5N3, H5N8, H5N9, H7N1, H7N2, H7N3, H7N4, 1-17N7, H9N2, and H1ON7 subtypes including the following subtypes commonly known as the “Spanish Flu,” “Asian Flu,” “Hong Kong Flu,” “Avian Flu,” “Swine Flu,” “Horse Flu,” and “Dog Flu.”

The term “subject,” as used herein, describes an organism, including humans and mammals, to which treatment with the compositions according to the present invention is provided. Mammalian species that benefit from the disclosed methods of treatment include, but are not limited to, apes, chimpanzees, orangutans, humans, monkeys; and domesticated animals (i.e., pets) such as dogs, cats, mice, rats, guinea pigs, and hamsters.

“Concurrent administration” and “concurrently administering,” as used herein, includes administering a compound or therapeutic method suitable for use with the methods of the invention (administration of a cysteamine compound) in the treatment of a Class I-V viral infection or for the treatment of Class I-V viral infection-related symptoms/complications.

As used herein, reference to a “cysteamine compound” includes cysteamine, the various cysteamine salts, which include pharmaceutically acceptable salts of a cysteamine compound, as well as prodrugs of cysteamine that can, for example, be readily metabolized in the body to produce cysteamine. Also included within the scope of the subject invention are analogs, derivatives, conjugates, and metabolic precursors (such as phosphocysteamine, cysteine, cystamine, pantethine, and the like) as well as metabolites (such as taurine, hypotaurine, and the like) of cysteamine, which have the ability as described herein to treat and/or prevent stress and stress-related symptoms/complications by lowering cortisol levels as well as augment immune activity. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

As contemplated herein, a cysteamine compound includes pantothenic acid. Pantothenic acid is a naturally occurring vitamin that is converted in mammals to coenzyme A, a substance vital to many physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

The term “pharmaceutically acceptable salt,” as used herein, refers to any salt of a cysteamine compound that is pharmaceutically acceptable and does not greatly reduce or inhibit the activity of the cysteamine compound. Suitable examples include acid addition salts, with an organic or inorganic acid such as acetate, tartrate, trifluoroacetate, lactate, maleate, fumarate, citrate, methane, sulfonate, sulfate, phosphate, nitrate, or chloride.

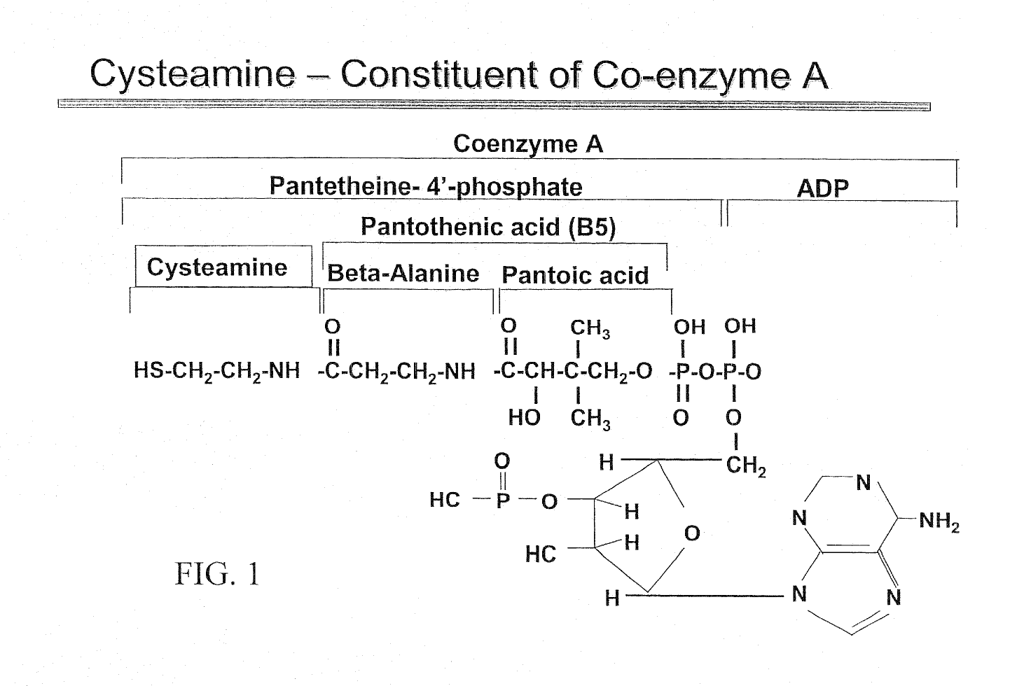

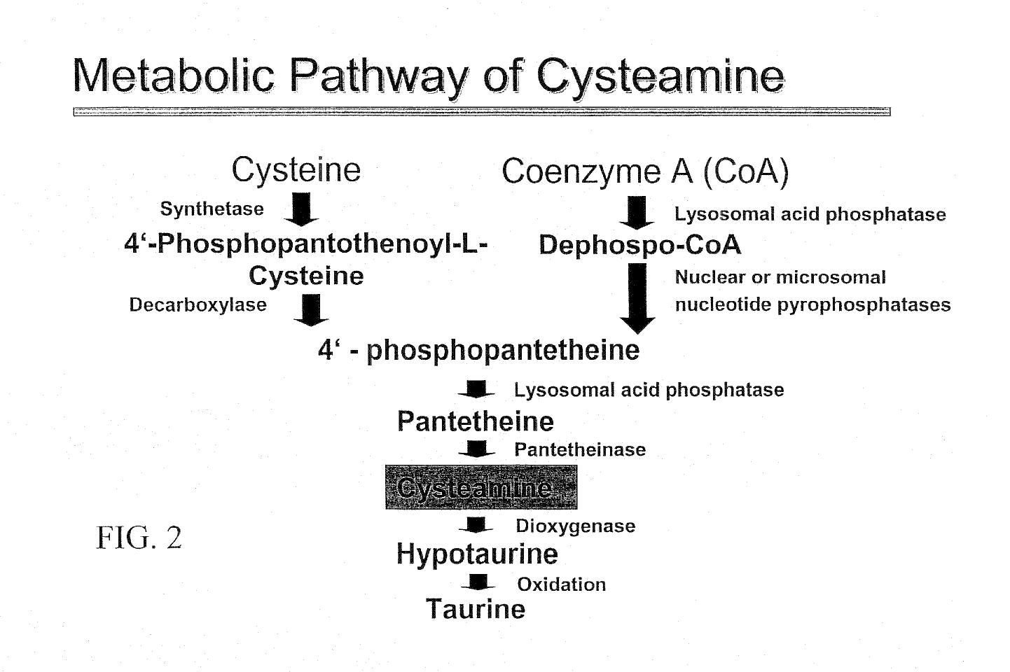

Accordingly, in one embodiment of the subject invention, the advantages of cysteamine, as set forth herein, can be achieved by promoting the endogenous production of cysteamine through natural metabolic process such as through the action of co-enzyme A or as a precursor and/or metabolite of cysteine (see FIGS. 1 and 2). This can be achieved by, for example, the administration of pantothenic acid.

The term “effective amount,” as used herein, refers to the amount necessary to elicit the desired biological response. In accordance with the subject invention, the effective amount of a cysteamine compound is the amount necessary to treat/prevent a Class I-V viral infection; treat/ameliorate symptoms associated with Class I-V viral infections; and/or prevent/delay/ameliorate the onset of complications associated with Class I-V viral infections. In a preferred embodiment, the effective amount of a cysteamine compound is the amount necessary to treat/prevent an influenza infection; treat/ameliorate symptoms associated with influenza infection; and/or prevent/delay/ameliorate the onset of complications in patients with increased risk for contracting complications associated with influenza infection. The amelioration in symptom and/or complication severity may be a 5%, 10%, 15%, 20%, 25% 30%, 35%, 40%, 45%, 50%, 55%, 60%, 65%, 70%, 75%, 80%, 85%. 90%, 95%, 98% or 99% decrease in severity.

As used herein, the term “Class I-V viruses” refers to the different classes of virus identified by genome composition and strategy for mRNA synthesis, as described in Lodish, H. et al., Molecular Cell Biology, Fourth Edition, W.H. Freeman and Company (2000). Class I-V viruses are identified as follows:
Class I viruses contain a single molecule of double-stranded DNA;
Class II viruses contain a single molecule of single-stranded DNA;
Class III viruses contain double-stranded genomic RNA;
Class IV viruses contain a single strand of viral mRNA (also known as a positive/plus strand of genomic RNA), wherein the viral mRNA encodes proteins and is infectious by itself; and
Class V viruses contain a single strand of an RNA sequence that is complimentary to the genomic viral mRNA (also known as a negative/minus strand of genomic RNA), wherein the genomic RNA acts as a template for synthesis of mRNA but does not itself encode proteins.

### Applications

The present invention provides materials and methods for treating and/or preventing a Class I-V viral infection through the administration of a cysteamine compound to a subject. Viral infections resulting from the following types of viruses are treated and/or prevented by administering a cysteamine compound as disclosed herein. The viruses include double-stranded DNA (dsDNA), single-stranded DNA (ssDNA), double-stranded genomic RNA (dsRNA), single-strand positive RNA, and single-strand negative RNA viruses. Contemplated viruses that can be treated in accordance with the subject invention include, but are not limited to, arboviruses (included but not limited to, dengue virus, yellow fever, and the like); adenoviruses (included but not limited to acute respiratory illness, pneumonia, conjunctivitis, gastroenteritis, pharyngitis, acute haemorrhagic cystitis, African swine fever, porcine circovirus, porcine adenoviruses A, B, and C); herpesviruses (included but not limited to herpes simplex virus, varicella zoster virus (chicken pox and shingles), Epstein-Barr virus); human papillomaviruses (included but not limited to HPV types 1-65); parvoviruses (included but not limited to parvovirus B19, canine parvovirus); reoviruses (included but not limited to orbivirus, rotavirus, aquareovirus, coltivirus); picornaviruses (included but not limited to enterovirus, rhinovirus, hepatovirus); coronaviruses (included but not limited to coronavirus and torovirus); flavivirus (included but not limited to petsivirus, hepatitis C-like viruses); togaviruses (included but not limited to alphavirus and rubivirus), orthomyxovirus (included but not limited to influenza A, B, and C viruses, avian influenza virus, Thogoto virus); bunyaviruses (included but not limited to Hantavirus, Nairovirus, phlebovirus); rhabdoviruses (included but not limited to rabies virus, ephemerovirus, vesiculovirus); and paramyxoviruses (included but not limited to measles virus and mumps virus).

The present invention is particularly applicable to non-human subject health, especially to non-human subjects infected with a Class I-V virus. For instance, the following, non-limited list of viruses and resultant conditions common in non-human subjects can be treated and/or prevented using the present invention: picornavirus (avian encephalomyelitis, duck hepatitis and calicivirus (cat) infections); orthomyxovirus (fowl plague and avian influenza (H5N1)); coronavirus (infectious bronchitis and coronaviral enteritis in poultry and canine corona virus in dogs); togavirus (pheasant encephalitis); paramyxovirus (Newcastle's Disease in poultry and canine distemper and parainfluenza in dogs); rhabdovirus (rabies and viral hemorrhagic disease in fish); and reovirus (poultry infectious bursal disease).

With regard to human subjects, the present invention is particularly applicable to the treatment and/or prevention of influenza virus infections, especially avian influenza virus infections. According to the subject invention, a cysteamine compound is useful in the treatment and/or prevention of various avian influenza strains, including viruses of subtype H1N1, H1N2, H2N2, H3N2, H3N8, H5N1, H5N2, H5N3, H5N8, H5N9, H7N1, H7N2, H7N3, H7N4, H7N7, H9N2, and H10N7. In one embodiment of the invention, cysteamine hydrochloride is administered to subjects (either human or animal) in order to treat and/or prevent a H5N1 avian influenza virus infection. The cysteamine hydrochloride can be administered alone or concurrently with other known agents known to be effective in treating and/or preventing an influenza infection.

In a related embodiment, a cysteamine compound (such as cysteamine hydrochloride) is administered alone or concurrently with other known agents that are used to treat and/or prevent an avian influenza viral (AIV) infection. The cysteamine compound can be administered to a subject via injection or oral administration.

Preferably, a dosage of at least 0.1 mg/mL of cysteamine hydrochloride, more preferably at least 1 mg/mL of cysteamine hydrochloride, and even more preferably at least 2 mg/mL of cysteamine hydrochloride, can be administered to a subject to treat and/or prevent a H5N1 AIV infection.

In certain preferred embodiments, the dosage of cysteamine hydrochloride administered in the treatment and/or prevention of an AIV infection (including viruses of subtype H1N1, H1N2, H2N2, H3N2, H3N8, H5N1, H5N2, H5N3, H5N8, H5N9, H7N1, H7N2, H7N3, H7N4, H7N7, H9N2, and H10N7) correlates to the concentration of virus present in the subject. More preferably, the dosage of cysteamine hydrochloride administered in the treatment and/or prevention of a H5N1 AIV infection correlates to a concentration of about LD50 of virus present in the subject.

### Concurrent Treatments

For a subject diagnosed with an influenza infection, a cysteamine compound can be concurrently administered with vaccinations, antiviral drugs, antitussives, mucolytics, and/or expectorants; antipyretics and analgesics; nasal decongestants.

By way of example, a compound can be provided in admixture with a cysteamine compound, such as in a pharmaceutical composition; or the compound and cysteamine can be provided as separate compounds, such as, for example, separate pharmaceutical compositions administered consecutively, simultaneously, or at different times. Preferably, if the cysteamine compound and the known agent (or therapeutic method) for treating/preventing influenza infection and/or treating influenza-related symptoms/complications are administered separately, they are not administered so distant in time from each other that the cysteamine compound and the known agent (method) cannot interact.

In certain embodiments of the invention, a cysteamine compound can be administered concurrently with, but not limited to, vaccination, antiviral medications such as amantadine, rimantadine, ribavirin, idoxuridine, trifluridine, vidarabine, acyclovir, ganciclovir, foscarnet, zidovudine, didanosine, zalcitabine, stavudine, famciclovir, oseltamivir, and valaciclovir (materials and/or methods used to treat an viral infection); or antitussives, mucolytics, and/or expectorants; antipyretics and analgesics; nasal decongestants (materials used to treat symptoms associated with an influenza infection).

By way of example, a compound for use with a cysteamine compound of the invention can be provided in admixture with the cysteamine compound, such as in a pharmaceutical composition. Alternatively, the compound and cysteamine can be provided as separate compounds, such as, for example, separate pharmaceutical compositions administered consecutively, simultaneously, or at different times. Preferably, if the cysteamine compound and the known agent (or therapeutic method) for treating/preventing influenza infection and/or treating influenza-related symptoms/complications are administered separately, they are not administered so distant in time from each other that the cysteamine compound and the known agent (method) cannot interact.

### Formulations

Compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The cysteamine compounds of the subject invention can be formulated according to known methods for preparing pharmaceutically useful compositions. Formulations are described in a number of sources, which are well known and readily available to those skilled in the art. For example, Remington's Pharmaceutical Science (Martin EW [1995] Easton Pa., Mack Publishing Company, 19^{th} ed.) describes formulations that can be used in connection with the subject invention.

The formulations may conveniently be presented in unit dosage form and may be prepared by any of the methods well known in the art of pharmacy. Such methods include the step of bringing into association the cysteamine compound with the carrier which constitutes one or more accessory ingredients. In general the formulations are prepared by uniformly and intimately bringing into association the cysteamine compound with liquid carriers or finely divided solid carriers or both, and then, if necessary, shaping the product.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

In accordance with the invention, compositions comprising, as an active ingredient, an effective amount of the cysteamine and one or more non-toxic, pharmaceutically acceptable carrier or diluent. Examples of such carriers for use in the invention include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents.

To provide for the administration of such dosages for the desired therapeutic treatment, compositions of the invention will typically comprise between about 0.1% and 95%, of the total composition including carrier or diluent. The dosage used can be varied based upon the age, weight, health, or the gender of the individual to be treated.

In one embodiment, the dosage of cysteamine administered to a patient to elicit a desired response is about 10 mg to about 3,000 mg per day. The desired response can include (1) prevention of Class I-V viral infections; preferably influenza infection; (2) a reduction in the severity, duration, or intensity of symptoms associated with Class I-V infections, preferably symptoms associated with influenza infection; and (3) prevention, delay, or reduction in the severity, duration, or intensity of complications related to a Class I-V viral infections, complications related to influenza infections. Preferably, cysteamine hydrochloride is administered daily at about 50 mg to 1,000 mg to elicit a desired response. In a more preferred embodiment, the dosage of cysteamine hydrochloride administered to a patient to elicit a desired response is about 200 mg to 900 mg per day.

### Pulmonary Administration

A preferred means of administration is through either a local administration to the lungs or nasal passage, e.g. into the respiratory tissues via inhalation, nebulization or intranasal administration.

Formulations for inhalation are well known in the art. Such formulation may include sterile aqueous solutions which may also contain buffers, diluents and other suitable additives, an example being PBS or Dextrose 5% in water.

The active compounds disclosed herein are preferably administered to the lung(s) or nasal passage of a subject by any suitable means. Active compounds may be administered by administering an aerosol suspension of respirable particles comprised of the active compound or active compounds, which the subject inhales. The active compound can be aerosolized in a variety of forms, such as, but not limited to, dry powder inhalants, metered dose inhalants, or liquid/liquid suspensions. The respirable particles may be liquid or solid. The particles may optionally contain other therapeutic ingredients.

The particulate pharmaceutical composition may optionally be combined with a carrier to aid in dispersion or transport. A suitable carrier such as a sugar (i.e., dextrose, lactose, sucrose, trehalose, mannitol) may be blended with the active compound or compounds in any suitable ratio (e.g., a 1 to 1 ratio by weight).

Particles comprised of the active compound for practicing the present invention should include particles of respirable size, that is, particles of a size sufficiently small to pass through the mouth or nose and larynx upon inhalation and into the bronchi and alveoli of the lungs. In general, particles ranging from about 1 to 10 microns in size (more particularly, less than about 5 microns in size) are respirable. Particles of non-respirable size which are included in the aerosol tend to deposit in the throat and be swallowed, and the quantity of non-respirable particles in the aerosol is preferably minimized. For nasal administration, a particle size in the range of 10-500 micons is preferred to ensure retention in the nasal cavity.

Liquid pharmaceutical compositions of active compound for producing an aerosol may be prepared by combining the active compound with a suitable vehicle, such as sterile pyrogen free water. The hypertonic saline solutions used to carry out the present invention are preferably sterile, pyrogen-free solutions, comprising from one to fifteen percent (by weight) of the physiologically acceptable salt, and more preferably from three to seven percent by weight of the physiologically acceptable salt.

Aerosols of liquid particles comprising the active compound may be produced by any suitable means, such as with a pressure-driven jet nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation.

Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate therapeutic aerosol generator. Aerosol generators for administering solid particulate therapeutics to a subject produce particles which are respirable and generate a volume of aerosol containing a predetermined metered dose of a therapeutic at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. in the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 200 ul, to produce a fine particle spray containing the active ingredient.

Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidant and suitable flavoring agents.

Administration can be provided by the subject or by another person, e.g., a caregiver. A caregiver can be any entity involved with providing care to the human: for example, a hospital, hospice, doctor's office, outpatient clinic; a healthcare worker such as a doctor, nurse, or other practitioner; or a spouse or guardian, such as a parent. The medication can be provided in measured doses or in a dispenser which delivers a metered dose.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A method for treating a viral infection, wherein said method comprises diagnosing a subject with the viral infection; and administering to the subject an effective amount of a cysteamine compound, wherein the administration is by pulmonary administration.

2. The method, according to claim 1, wherein the viral infection is selected from the group consisting of avian influenza viruses; adenoviruses; herpesviruses; human papillomaviruses; parvoviruses; reoviruses; picornaviruses; coronaviruses; flavivirus; togaviruses, orthomyxovirus; bunyaviruses; rhabdoviruses; and paramyxoviruses.

3. The method, according to claim 2, wherein the subject is infected with at least one of the group consisting of avian influenza virus, pneumonia, conjunctivitis, gastroenteritis, pharyngitis, acute haemorrhagic cystitis, herpes simplex virus, varicella zoster virus, Epstein-Barr virus, HPV types 1-65, parvovirus B19, canine parvovirus, orbivirus, rotavirus, aquareovims, coltivirus, enterovirus, rhinovirus, hepatovirus, coronavirus and torovirus, petsivirus, hepatitis C-like viruses, alphavirus, rubivirus, influenza A, B, and C viruses, Thogoto virus, Hantavirus, Nairovirus, phlebovirus, rabies virus, ephemerovirus, vesiculovirus, measles virus, and mumps virus.

4. The method, according to claim 1, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

5. The method, according to claim 4, wherein said cysteamine salt is cysteamine hydrochloride.

6. The method, according to claim 1, wherein said administration is by inhalation.

7. A composition comprising an effective amount of a cysteamine compound for preventing a viral infection and a pharmaceutical carrier, wherein said composition is formulated for pulmonary administration.

8. The method, according to claim 7, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

9. The method, according to claim 8, wherein said cysteainine salt is cysteamine hydrochloride.
